(19) United States
(12) Patent Application Publication
(10) Pub. No.: US 2021/0217522 A1
(43) Pub. Date: Jul. 15, 2021

(54) MACHINE LEARNING MODEL FOR SURFACING SUPPORTING EVIDENCE

(71) Applicant: Clover Health, Jersey City, NJ (US)

(72) Inventors: Peter Vladimir Loscutoff, Berkeley, CA (US); Christopher James Lauinger, Golden, CO (US); Melanie Goetz, Oakland, CA (US); Robert Tristan Williams, New York, NY (US); Emily Anderson, San Mateo, CA (US)

(21) Appl. No.: 16/742,750

(22) Filed: Jan. 14, 2020

(51) Int. Cl. G16H 50/20; G16H 10/60; G16H 40/67; G16H 70/40; G16H 40/20; G06N 20/00

(52) U.S. Cl. CPC G16H 50/20; G16H 10/60; G06N 20/00; G16H 70/40; G16H 40/20; G16H 40/67

(57) ABSTRACT

Systems and methods including analyzing profiles, generating recommendation(s) and supporting evidence associated with the recommendation(s) related to medical services provided to a patient, and transmitting the recommendation(s) and supporting evidence associated with the recommendation(s) to a device that displays the information are disclosed. The supporting evidence may be presented based on a statistical relevance of the information and/or a likelihood that a medical professional will utilize the information.

### BACKGROUND

Doctors, nurses, or other medical professionals often examine patients to determine health related issues. Examination(s) may include in-person visits (e.g., hospital or in-home), over the phone, and/or virtually. During the examination, the medical professionals may be provided information associated with the patient. Determining where this information is sourced and what information to present to the medical professional, for instance, may be important in properly diagnosing a patient and/or identifying future measures to take. Described herein are improvements in technology and solutions to technical problems that may be used, among other things, to increase the materiality of patient examinations.

### BRIEF DESCRIPTION OF THE DRAWINGS

The detailed description is set forth below with reference to the accompanying figures. In the figures, the left-most digit(s) of a reference number identifies the figure in which the reference number first appears. The use of the same reference numbers in different figures indicates similar or identical items. The systems depicted in the accompanying figures are not to scale and components within the figures may be depicted not to scale with each other.

FIG. 1 illustrates an example of dynamically surfacing supporting evidence in an environment. The environment may include a medical professional and a patient, whereby the medical professional receives information and/or supporting evidence associated with the information displayed by a device. In some instances, the device may receive the information and/or the supporting evidence associated with the information from remote computing resource(s).

FIG. 2 illustrates a block diagram of selected functional components of the computing resource(s) of FIG. 1.

FIG. 3 illustrates a block diagram of selected functional components of the device of FIG. 1.

FIG. 4 illustrates a flow diagram of an example process of the remote computing resource(s) of FIG. 1 surfacing supporting evidence.

FIG. 5 an example process of updating the device of FIG. 1 to display information and/or supporting evidence associated with the information.

### DETAILED DESCRIPTION

Systems and methods of dynamically surfacing supporting evidence associated with a recommendation (e.g., potential diagnosis, gap in medical coverage, recommended medication, etc.) to a medical professional are described herein. In diagnosing a patient with an illness, disease, condition, or sickness, medical professionals (e.g., doctor, nurse, physician's assistant, nurse practitioner, etc.) may receive information and/or recommendations based on user profiles that are associated with individual patients. Charts detailing a patient's medical history may be used in diagnosing, such as results from tests (e.g., blood tests, Electrocardiography (EKG), etc.). However, a recommendation (e.g., potential diagnosis, gap in medical coverage, recommended medication, etc.) to a medical professional may not carry much weight if the medical professional is not aware of how the recommendation was formulated and what information was used to generate the recommendation. For instance, during a patient visit, a medical professional may access a service that provides recommendations based on a user profile associated with the patient. In one example, the service may recommend to the medical professional to diagnose the patient with a disease (e.g., diabetes) based on medical history information stored in the user profile. The medical professional may not know how accurate the recommendation is, or may not trust the recommendation, without knowing the information that was used to generate the recommendation. In some cases, the medical professional may have access to the information used to generate the recommendation, but the information may not be efficiency organized such that the medical professional can quickly find the information most relevant to the situation and/or diagnosis. As a result of the foregoing, medical professionals may ignore recommendations for patient care and/or may improperly correlate certain symptoms with diagnoses, potentially leading to a misdiagnosis or a failure to diagnose.

In light of the above, the present application describes techniques for surfacing supporting evidence associated with recommendation(s) (e.g., potential diagnosis, gap in medical coverage, recommended medication, etc.) to a medical professional when examining and/or interacting with a patient. The recommendation(s) and supporting evidence may be provided to a device operated by the medical professional, such as a tablet, computer, or phone, and the device may be configured to display the recommendation(s) as well as the supporting evidence. The medical professional may then make a determination regarding the accuracy of the recommendation(s) based on the supporting evidences associated with the recommendation(s). Thereafter, feedback may be entered on the device. In some instances, this feedback may indicate which of a plurality of data included in the supporting evidence the medical professional used in order to make a determination regarding the accuracy of the recommendation(s). For instance, the recommendation may include a potential diagnosis in which the patient is suspected of having diabetes. The medical professional may interact with the remote device to select the diabetes diagnosis and the remote device may present supporting evidence that was used to determine that the patient may have diabetes. In some instances, the supporting evidence may include test results, medical history, personal information, identifying information associated with test results (e.g., a name of a company performing the tests), etc. The medical professional may provide feedback by selecting which test results were utilized to determine that the diagnosis is accurate. In some instance, the feedback may be stored in a medical professional profile maintained by a remote computing resource(s). The remote computing resource(s) may determine which information to include in the supporting evidence, how information is arranged in the supporting evidence, and/or which information is emphasized (e.g., highlighted, bolded, italicized, underlined, etc.) in the supporting evidence, based on information stored in the medical professional profiles (e.g., historical records of medical professional actions).

The recommendation(s) and/or the supporting evidence associated with the recommendation(s) displayed on the device may be received and/or generated from a remote computing resource(s) (e.g., cloud, server, etc.) and the recommendation(s) and/or the supporting evidence may be tailored according to the patient's medical history, symptoms, and/or personal information as well as the medical professionals historical records. As an example, the remote computing resource(s) may include (e.g., store) user profiles corresponding to patients and/or one or more databases associated with medical records, news, diagnostics, statistics, and/or other medical information. The remote computing resource(s) may also store medical professional profiles corresponding to medical professionals and/or one or more databases associated with medical records, news, diagnostics, statistics, and/or other medical information associated with previous appointments involving the medical professional. The remote computing resource(s) may analyze the user profiles, such as a medical history of the patient, and/or the one or more databases to determine recommendation(s) to present to the medical professional. Additionally, the remote computing resource(s) may analyze the medical professional profile, such as a historical record of utilized information, and/or the one or more databases to determine what supporting evidence to present to the medical professional. In some instances, the recommendation may relate to one or more suspected diagnoses of the patient, recommended medication for the patient, and/or a gap in medical coverage recommendation for the patient and the supporting evidence may include one or more test results, medical history, personal information, or identifying information associated with test results (e.g., a name of a company performing the test.

The remote computing resource(s) may employ machine learning algorithms or techniques to generate the recommendation(s) and/or the supporting evidence associated with the recommendation(s). In some instances, the machine leaning techniques may correlate a patient's medical history or historical trends with one or more recommendations of the patient, despite, in some instances, the patient's medical history (or other information) failing to indicate the suspected diagnoses. More specifically, while a patient's medical history may include symptoms associated with an illness, these symptoms, individually, may not be correlated to a suspected diagnosis. In this sense, the machine learning techniques function to aggregate and analyze trends in a patient's medical history as well as, in examples, trends in other patients' medical histories, to determine one or more suspected diagnoses, or other recommendations.

The device may display the recommendation(s) for the medical professional to utilize when examining a patient. For instance, after analyzing the user profiles and/or the databases, the recommendation(s) may relate to one or more potential suspected diagnoses of the patient (e.g., diabetes, heart disease, etc.), potential gaps in coverage associated with the patient, and/or a recommended prescription for the patient. While the medical professional is examining the patient, the medical professional may make an assessment as to whether the patient has the one or more suspected diagnoses, is, in fact, having a gap in medical coverage, and/or requires the recommended medication. That is, the medical professional may review the supporting evidence associated with the recommendation(s) to determine the accuracy of the recommendation(s). In making this assessment, the medical professional may ask questions, perform tests, and so forth before providing an indication as to the suspected diagnoses, potential gaps in coverage, and/or a recommended prescription.

In some instances, the remote computing resources(s) may determine a statistical relevance of the supporting evidence used to generate the recommendation(s). For example, the remote computing resource(s) may store, or have access to, the information that was used to determine which recommendation to provide the medical professional. In some instances, some of the information may be more relevant than others. For example, if the recommendation includes a potential diagnosis, such as diabetes, then the remote computing resource(s) may determine that a particular test, such as a blood sugar test, performed on the patient is more relevant than a different test performed on the patient, such as a skin biopsy. The remote computing resource(s) may determine the statistical relevance of the information used to determine the recommendation(s) by comparing the recommendation(s) and information used to determine recommendation(s) to previous recommendation(s) and previous information used to determine recommendation(s). In some instances, the remote computing resource(s) may access a medical professional profile and determine which types of information (i.e., supporting evidence) that a particular medical professional commonly uses to determine if a recommendation is accurate. This may be done by receiving feedback from the medical professional indicating which information included in the supporting evidence was used to determine if the recommendation(s) is accurate. In some instances, the remote computing resources(s) may utilize a machine learning model to determine which information is most statistically relevant by determining a confidence score of the recommendation. For example, a recommendation based off a first test, a second test, and a third test may result in a 95% confidence score of the recommendation, via a machine learning model. The remote computing resource(s) may determine that removal of the third test from the machine learning model results in a 94% confidence score of the recommendation (i.e., the recommendation being based off of the first test and the second test) and removal of the second confidence score results in a 50% confidence score of the recommendation (i.e., the recommendation being based off of the first test and the third test). The remote computing resource(s) may then determine that the second test is more statistically relevant than the third test due to the effect it has on the confidence score of the recommendation. In some instances, removal of a single particular test may have a minimal effect on the confidence score of the recommendation, but removal of multiple tests may have a substantial effect on the confidence score of the recommendation. In this case, the remote computing resource(s) may determine that the multiple tests are substantially equally statistically relevant.

In some instances, the remote computing resource(s) may determine a likelihood that a medical professional will use the supporting evidence associated with the recommendation. For example, the remote computing resource(s) may determine a statistical relevance of the supporting evidence and may determine the likelihood that the medical professional will utilize the supporting evidence based on the statistical relevance of the supporting evidence. In some cases, the remote computing resource(s) may present the supporting evidence to the medical professional based on determining the likelihood that the supporting evidence will be utilized. For example, the supporting evidence may be presented in an order listed from most likely to be utilized to least likely to be utilized (e.g., in the case of a diabetes diagnosis, present a blood sugar test ahead of a skin biopsy test). That is, the remote computing resource(s) may rank the supporting evidence based on a likelihood that the supporting evidence will be utilized and present the supporting evidence in a list based on the ranking. In some cases, the ranking may be based on the statistical relevance of each piece of supporting evidence. In some cases, the remote computing resource(s) may cause the remote device to emphasize (e.g., highlighted, bolded, italicized, underlined, etc.) supporting evidence that is more likely to be utilized. In this way, the medical professional can quickly determine if the recommendation(s) provided are accurate and the medical professional can efficiently and swiftly attend to the patient.

In some instances, the device may transmit data corresponding to which supporting evidence associated with the recommendation was used to the remote computing resource(s). The data may be analyzed by the remote computing resource(s) and utilized to analyze trends for future diagnoses suspected in additional patients and may be used to update the medical professional profiles to determine future statistically relevant information and/or likelihoods that the medical professional will utilize the information. For instance, the machine learning techniques may analyze the data to determine that a particular medical professional prefers information from a certain test provider over another and may subsequently present supporting evidence from the preferred provider before any supporting evidence from the other provider.

Given the communicative relationship between device and the remote computing resource(s), the recommendations and supporting evidence associated with the recommendation may be updated in real time and according to the data transmitted from the device to the remote computing resource. That is, the device may transmit the data and may receive, substantially contemporaneously with transmitting the data, updated recommendation(s) and supporting evidence associated with the recommendation. In other words, the remote computing resource(s) may continuously generate and transmit, substantially contemporaneously with receiving the data, updated recommendation(s) and supporting evidence associated with the recommendation. In doing so, using the machine learning techniques described herein, the recommendation(s) and supporting evidence associated with the recommendation(s) may be refined according to the data provided by the medical professional, thereby assisting in determining or helping to refine one or more suspected diagnoses of the patient. In some instances, the device may transmit one or more data individually, or the data may be transmitted as a batch.

With the above process, the device and the remote computing resource(s) may be in communication to generate recommendation(s) and supporting evidence associated with the recommendation(s). After a sufficient amount of recommendation(s) and supporting evidence associated with the recommendation(s) are generated and after a sufficient amount of data from the medical professionals indicating which supporting evidence is utilized is received, the remote computing resource(s) (or the device), may refine the process of determining statistical relevancies for supporting evidence as well as a likelihood of which supporting evidence will be utilized. In some instances, determining which supporting evidence to present and/or which supporting evidence to emphasize may be determined after a threshold amount of recommendation(s) and supporting evidence associated with the recommendation(s) are presented, after a threshold amount of data from the medical professional is received, after a confidence or probability level of the supporting evidence exceeds a threshold, and/or any combination thereof.

Compared to conventional techniques, which include predefined or static recommendation(s), or fail to provide supporting evidence for recommendation(s), the process described herein provides for the real-time generation and transmittal of recommendation(s) and supporting evidence associated with the recommendation(s). Such real-time information is crucial given the time-sensitive interaction with patients and the time-sensitive nature of diagnosing patients. In other words, as medical professionals often have limited time with patients, the recommendation(s) and supporting evidence associated with the recommendation(s) generated must be generated substantially quickly and organized efficiently such that the medical professional can quickly identify the relevant information. By way of comparison, if the information is simply listed alphabetically or organized in an order that the tests were performed, the medical professional may not see the most relevant supporting evidence associated with the recommendation and the medical professional may not fully trust or understand the recommendation, resulting in inefficiency's that may potentially harm the patient. Instead the system and methods described herein allow for the time-sensitive generation and transmittal of recommendation(s) and supporting evidence associated with the recommendation(s). Moreover, through analyzing the user profiles, medical professional profiles, and the databases, the instant application allows for identification of statistically relevant supporting evidence and determinations of which supporting evidence a particular medical professional is likely to utilize in determining if the recommendation is relevant. The analysis performed by the machine learning technique in generating trends, historical models, comparing user profiles, comparing medical professional profiles, comparing database(s) would not otherwise be possible in conventional methods given the vast amount of information that is required to be analyzed in such a time-sensitive manner.

The present disclosure provides an overall understanding of the principles of the structure, function, manufacture, and use of the systems and methods disclosed herein. One or more examples of the present disclosure are illustrated in the accompanying drawings. Those of ordinary skill in the art will understand that the systems and methods specifically described herein and illustrated in the accompanying drawings are non-limiting embodiments. The features illustrated and/or described in connection with one embodiment may be combined with the features of other embodiments, including as between systems and methods. Such modifications and variations are intended to be included within the scope of the appended claims. Additional details are described below with reference to several example embodiments.

### Illustrative Environment

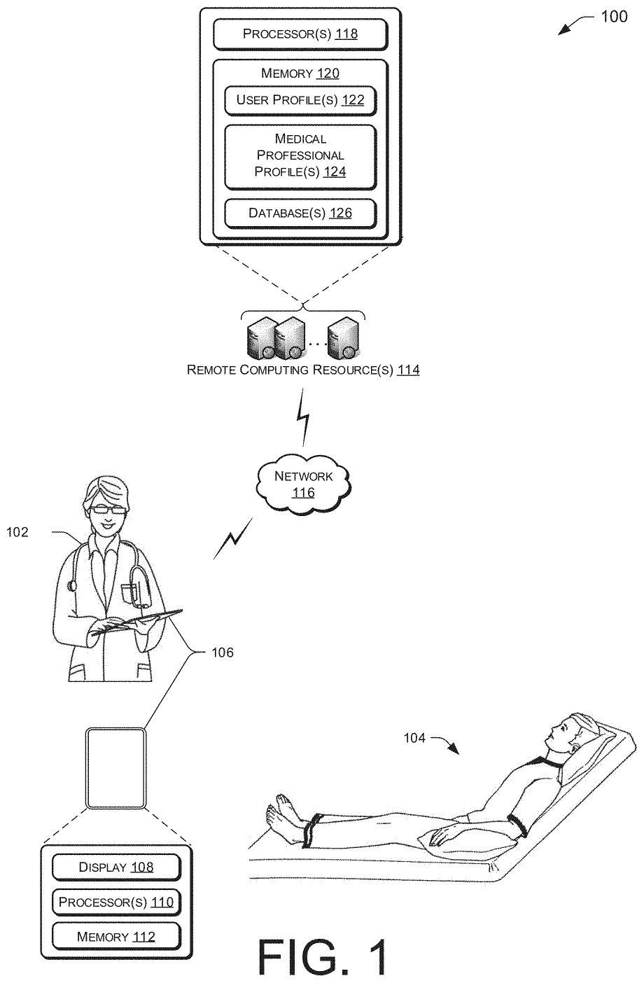

FIG. 1 shows an illustrative environment 100 which may include a provider 102 and a patient 104. In some instances, the environment 100 may be located at a medical facility (e.g., hospital, clinic, etc.) or at a residence of the patient 104. The environment 100 may also include a device 106 with which the provider 102, or in some instances, the patient 104 may interact. In the illustrative implementation, the provider 102 is holding the device 106. In other implementations, the patient 104 may hold the device 106. Further, more than one device 106 may be included within the environment 100. For instance, the provider 102 may have a device 106 while the patient 104 may have a separate device 106. In such instances, the devices may be configured to communicate with one another.

The device 106 may include a display 108 to display content. In some instance, the display 108 may include a touchscreen capable of receiving input from the provider 102 (or the patient 104). For instance, the display 108 may include a graphical user interface (GUI) that receives input from the provider 102. The display 108 may also include a virtual keyboard, buttons, input fields, and so forth, to permit the provider 102 to interact with the device 106.

The device 106 includes processor(s) 110 and memory 112. Discussed in detail herein, the processor(s) 110 may configure the device 106 to present recommendation(s) and supporting evidence associated with the recommendation(s) on the display 108. Therein, the provider 102 may determine an accuracy of the recommendation(s) based on the supporting evidence and address the patient 104, may perform examination(s) or diagnostics related to the recommendation(s) and supporting evidence associated with the recommendation(s), and/or enter an input on the device 106. For instance, FIG. 1 illustrates the provider 102 interacting with the patient 104. The device 106 may present a recommendation (e.g., potential diagnosis, gap in medical coverage, recommended medication, etc.) as well as supporting evidence associated with the recommendation (e.g., test results, medical history, personal information, identifying information associated with test results, a name of a company performing the tests, etc.). The provider 102 may select one of the supporting evidence listed on the display 108 as being of particular relevance in determining that the recommendation is accurate. The input received by the device 106 may be transmitted to the remote computing resource 114.

The device 106 may be communicatively coupled to one or more remote computing resource(s) 114 to receive the recommendation(s) and supporting evidence associated with the recommendation(s). Additionally, the device 106 may transmit inputs from the provider 102 to the remote computing resource(s) 114. The remote computing resource(s) 114 may be remote from the environment 100 and the device 106. For instance, the device 106 may communicatively couple to the remote computing resource(s) 114 over a network 116. In some instances, the device 106 may communicatively couple to the network 116 via wired technologies (e.g., wires, USB, fiber optic cable, etc.), wireless technologies (e.g., RF, cellular, satellite, Bluetooth, etc.), or other connection technologies. The network 116 is representative of any type of communication network, including data and/or voice network, and may be implemented using wired infrastructure (e.g., cable, CATS, fiber optic cable, etc.), a wireless infrastructure (e.g., RF, cellular, microwave, satellite, Bluetooth, etc.), and/or other connection technologies.

The remote computing resource(s) 114 may be implemented as one or more servers and may, in some instances, form a portion of a network-accessible computing platform implemented as a computing infrastructure of processors, storage, software, data access, and so forth that is maintained and accessible via a network such as the Internet. The remote computing resource(s) 114 do not require end-user knowledge of the physical location and configuration of the system that delivers the services. Common expressions associated with these remote computing resource(s) 114 may include “on-demand computing,” “software as a service (SaaS),” “platform computing,” “network-accessible platform,” “cloud services,” “data centers,” and so forth.

The remote computing resource(s) 114 include a processor(s) 118 and memory 120, which may store or otherwise have access to one or more user profile(s) 122, one or more medical professional profile(s) 124, and/or one or more database(s) 126. Discussed in detail herein, the remote computing resource(s) 114 may generate and transmit the recommendation(s) and supporting evidence associated with the recommendation(s) to the device 106 and in generating the recommendation(s) and supporting evidence associated with the recommendation(s), the remote computing resource(s) 114 may utilize the user profile(s) 122, the medical professional profiles, and/or the database(s) 126. In some cases, the one or more user profile(s) 122, one or more medical professional profile(s) 124, and/or one or more database(s) 126 may store a time in which patient 104 is scheduled to have an appointment and the remote computing resource(s) 114 may transmit the recommendation(s) and/or supporting evidence associated with the recommendation(s) to the device 106 at the given scheduled time.

As used herein, a processor, such as processor(s) 110 and/or 118, may include multiple processors and/or a processor having multiple cores. Further, the processors may comprise one or more cores of different types. For example, the processors may include application processor units, graphic processing units, and so forth. In one implementation, the processor may comprise a microcontroller and/or a microprocessor. The processor(s) 110 and/or 118 may include a graphics processing unit (GPU), a microprocessor, a digital signal processor or other processing units or components known in the art. Alternatively, or in addition, the functionally described herein can be performed, at least in part, by one or more hardware logic components. For example, and without limitation, illustrative types of hardware logic components that may be used include field-programmable gate arrays (FPGAs), application-specific integrated circuits (ASICs), application-specific standard products (ASSPs), system-on-a-chip systems (SOCs), complex programmable logic devices (CPLDs), etc. Additionally, each of the processor(s) 110 and/or 118 may possess its own local memory, which also may store program components, program data, and/or one or more operating systems.

The memory 112 and/or 120 may include volatile and nonvolatile memory, removable and non-removable media implemented in any method or technology for storage of information, such as computer-readable instructions, data structures, program component, or other data. Such memory 112 and/or 120 may include, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, RAID storage systems, or any other medium which can be used to store the desired information and which can be accessed by a computing device. The memory 112 and/or 120 may be implemented as computer-readable storage media (“CRSM”), which may be any available physical media accessible by the processor(s) 110 and/or 118 to execute instructions stored on the memory 112 and/or 120. In one basic implementation, CRSM may include random access memory (“RAM”) and Flash memory. In other implementations, CRSM may include, but is not limited to, read-only memory (“ROM”), electrically erasable programmable read-only memory (“EEPROM”), or any other tangible medium which can be used to store the desired information and which can be accessed by the processor(s).

### Illustrative Remote Computing Resources

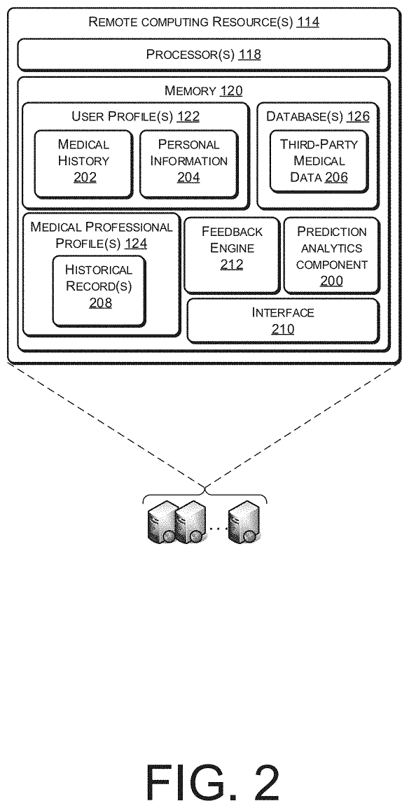

FIG. 2 shows selected functional components of the remote computing resource(s) 114. The remote computing resource(s) 114 includes the processor(s) 118 and the memory 120. As illustrated, the memory 120 of the remote computing resource(s) 114 stores or otherwise has access to user profile(s) 122, the medical professional profiles 124, the database(s) 126, and a prediction analytics component 200. The user profile(s) 122 may correspond to a respective user (e.g., patients). Each user profile 122 may include a user's medical history 202 and personal information 204. In some instances, the medical history 202 may include a medical history of the user, such as diagnoses (e.g., disease, illness, etc.), treatments (e.g., medications, surgeries, therapy, etc.), family medical history (e.g., diabetes, Alzheimer's, etc.), measurements (e.g., weight, height, etc.), symptoms (e.g., sore throat, back pain, loss of sleep, etc.), and so forth. The personal information 204 may include names (e.g., social security number (SSN)), identifiers, residence, work history, acquaintances, heritage, age, and so forth. The medical history 202 and/or the personal information 204 may be received using record locators and/or searching databases.

The database(s) 126 may include information or third-party medical data 206 obtained from third-party sources. The third-party sources may include a source (or service) that collects, stores, generates, filters, and/or provides medical news. In some instances, the third-party sources that provide the third-party medical data 206 may include news agencies, governmental agencies or services (e.g., U.S. Department of Health and Human Services (HHS), Centers for Disease Control and Prevention (CDC), National Institute of Health (NIH), etc.), medical new websites or sources (e.g., webmd.com, etc.), other medical sources (e.g., American Red Cross, Universities, Hospitals, etc.). The third-party medical data 206 may also include data obtained from other online resources that search for content, such as medical information. For instance, the online resources may include, but are not limited to, search engines (e.g., GOOGLE®), social media sites (e.g., FACEBOOK®, INSTRAGRAM®, etc.), databases, and/or other online resources. The remote computing resource(s) 114 may be in communication with the third-party sources to obtain, retrieve, and/or receive the third-party medical data 206 representing medical situations, medical conditions, and/or medical news.

As noted above, the remote computing resource(s) 114 may analyze the user profile(s) 122 and/or the database(s) 126 to generate recommendation(s) and supporting evidence associated with the recommendation(s) for a patient. For instance, the prediction analytics component 200 may analyze the user profile(s) 122, the medical professional profile(s) 124, and/or the database(s) 126 to determine recommendation(s) and supporting evidence associated with the recommendation(s). The prediction analytics component 200 may also be configured to determine a statistical relevance of individual data included in the supporting relevance and a likelihood that a particular medical professional, such as provider 102, will utilize the supporting evidence when determining the accuracy of the recommendation. Stated alternatively, the prediction analytics component 200 functions to determine recommendations, such as suspected diagnoses of the patient (e.g., diabetes, heart disease, etc.), potential gaps in coverage (e.g., mammograms) associated with the patient, and/or a recommended prescription for the patient that should be asked of the patient in determining one or more suspected health concerns (or diagnoses) of the patient or whether the patient is suspected of having particular diagnoses. For instance, based on analyzing the user profile(s) 122, the medical professional profile(s) 124, and/or the database(s) 126, the prediction analytics component 200 may identify suspected diagnoses of the patient. In some instances, the analysis may involve comparing symptoms stored in the user profile(s) 122 to the database(s) 126 (or other user profile(s) 122) to determine correlations between the patient's symptoms and one or more suspected diagnoses. That is, continuing with the above example, based on the analysis, the prediction analytics component 200 may determine that symptoms of a patient correlate closely with one or more diagnoses.

Additionally, or alternatively, the prediction analytics component 200 may determine the suspected diagnoses despite the user profile(s) 122 failing to indicate such diagnoses. For instance, the user profile 122 of a patient may indicate two distinct symptoms, such as a first symptom (e.g., high blood sugar levels) and a second symptom (e.g., skin infections). These symptoms may be analyzed by the prediction analytics component 200 to determine that the patient is suspected of having diabetes. However, taken individually, these symptoms may fail to indicate that diabetes is a suspected diagnosis. In other words, individually, the first symptom and the second symptom may not indicate that the patient has diabetes and/or the first symptom and the second symptom may not indicate the probability of the suspected diagnosis over a threshold. Using the prediction analytics component 200, the symptoms of a patient may be aggregated and correlated to symptoms associated with a suspected diagnosis (e.g., diabetes). That is, when looked at collectively, the prediction analytics component 200 may determine that the first symptom and the second symptom may be indicative of diabetes. Using this determination, the prediction analytics component 200 may determine a statistical relevance of the information (i.e., supporting evidence) used to make the recommendation(s).

The recommendation(s) generated are a result of the outcomes of the prediction analytics component 200. Predictive analytic techniques may include, for example, predictive questioning, machine learning, and/or data mining. Generally, predictive questioning may utilize statistics to predict outcomes and/or question(s) to propose in future. Machine learning, while also utilizing statistical techniques, provides the ability to improve outcome prediction performance without being explicitly programmed to do so. Any number of machine learning techniques may be employed to generate and/or modify the recommendation(s) describes herein. Those techniques may include, for example, decision tree learning, association rule learning, artificial neural networks (including, in examples, deep learning), inductive logic programming, support vector machines, clustering, Bayesian networks, reinforcement learning, representation learning, similarity and metric learning, sparse dictionary learning, and/or rules-based machine learning.

Information from stored and/or accessible data (e.g., the medical history 202, the personal information 204, and/or the third-party medical data 206, etc.) may be extracted from the user profile(s) 122, the medical professional profile(s) 124, and/or the database(s) 126 and utilized by the prediction analytics component 200 to predict trends and behavior patterns. The predictive analytic techniques may be utilized to determine associations and/or relationships between explanatory variables and predicted variables from past occurrences and utilizing these variables to predict the unknown outcome. The predictive analytic techniques may include defining the outcome and data sets used to predict the outcome. In defining the outcome, the prediction analytics component 200 may identify or determine supporting evidence (i.e., data sets) that was used to generate the recommendation (i.e., the outcome). Data analysis may include using one or more models, including for example one or more algorithms, to inspect the data with the goal of identifying useful information and arriving at one or more determinations that assist in predicting the outcome of interest. One or more validation operations may be performed, such as using statistical analysis techniques, to validate accuracy of the models. Thereafter predictive modelling may be performed to generate accurate predictive models for future events. By so doing, the prediction analytics component 200 may utilize data from the user profile(s) 122, the medical professional profiles 124, and/or the database(s) 126, as well as features from other systems as described herein, to generate a recommendation (e.g., predict or otherwise determine a probability of one or more suspected diagnoses, predict or otherwise determine a gap in medical coverage, and/or recommend a medication). Certain variables (e.g., symptoms) of the patient may be weighed more heavily than other symptoms in determining the outcome. Outcome prediction may be deterministic such that the outcome is determined to occur or not occur. Additionally, or alternatively, the outcome prediction may be probabilistic of whether the outcome is determined to occur to a certain probability and/or confidence.

Importantly, in utilizing outcomes of the prediction analytics component 200, the processor(s) 118 may determine a statistical relevance of the supporting evidence used to generate the recommendation(s). For example, the remote computing resource(s) 118 may store, or have access to, the information that was used to determine which recommendation to provide the provider 102 (e.g., data from the user profile(s) 122, the medical professional profiles 124, and/or the database(s) 126). In some instances, some portions of the information may be more relevant than others for determining an accurate recommendation. For example, if the recommendation includes a potential diagnosis, such as diabetes, then the prediction analytics component 200 may determine that a particular test, such as a blood sugar test, performed on the patient 104 is more relevant than a different test performed on the patient 104, such as a skin biopsy. In some cases, the prediction analytics component 200 may determine the statistical relevance of the information used to determine the recommendation(s) by comparing the recommendation(s) and information used to determine recommendation(s) to previous recommendation(s) and previous information used to determine recommendation(s). In some instances, the prediction analytics component 200 may access the medical professional profile 124 and a historical record(s) 208 and determine which types of information (i.e., supporting evidence) that a particular medical professional commonly uses to determine if a recommendation is accurate. This may be done by receiving feedback from the medical professional indicating which information included in the supporting evidence was used to determine if the recommendation(s) is accurate. In some instances, the prediction analytics component 200 may utilize a machine learning model to determine which information is most statistically relevant by determining a confidence score of the recommendation. For example, a recommendation based off a first test, a second test, and a third test may result in a 95% confidence score of the recommendation, via a machine learning model. The prediction analytics component 200 may determine that removal of the third test from the machine learning model results in a 94% confidence score of the recommendation (i.e., the recommendation being based off of the first test and the second test) and removal of the second confidence score results in a 50% confidence score of the recommendation (i.e., the recommendation being based off of the first test and the third test). The remote computing resource(s) 114 may then determine that the second test is more statistically relevant than the third test due to the effect it has on the confidence score of the recommendation. In some instances, the remote computing resource(s) 114 may determine that one of the supporting evidence is more statically relevant based on a degree of change that the supporting evidence has on the confidence score of the recommendation. For example, removal or addition of one of the supporting evidence may cause the confidence score of the recommendation to drop or rise above a predefined threshold and the remote computing resource(s) 114 may then determine a statistical relevance of the removed or added supporting evidence. In some instances, removal of a single particular test may have a minimal effect on the confidence score of the recommendation, but removal of multiple tests may have a substantial effect on the confidence score of the recommendation. In this case, the prediction analytics component 200 may determine that the multiple tests are substantially equally statistically relevant.

In some cases, the recommendation(s) and/or the supporting evidence associated with the recommendation(s) may be transmitted to the device 106 in response to a pull request from the device 106. Additionally, or alternatively, the recommendation(s) and/or the supporting evidence associated with the recommendation(s) may be pushed to the device 106 after generating the recommendation(s) and/or the supporting evidence associated with the recommendation(s). The remote computing resource(s) 114 may transmit the recommendation(s) and/or the supporting evidence associated with the recommendation(s) with a command that causes the device 106 to display the recommendation(s) and/or the supporting evidence associated with the recommendation(s). To communicate with the device 106, the third-party sources providing the third-party data 206, or other entities, the remote computing resource(s) 114 include an interface 210. In some cases, the supporting evidence that was used to generate the recommendation may be in the form of raw data that may not be usable to be presented for a user. In this case, the remote computing resource(s) 114 may alter and/or process the raw data such that it is presentable to a medical professional. For example, the data received from the database 126, the user profile(s) 122, and/or the medical professional profile(s) 124 may be in the form of raw data that the prediction analytics component 20 uses to determine a recommendation. The remote computing resource(s) 114 may determine which data that was used from the database 126, the user profile(s) 122, and/or the medical professional profile(s) 124 will be used as the supporting evidence associated with the recommendation and may process the data to be presented via a medical professionals device, such as device 106. This may include processing the data into text, image, etc.

In some instances, the remote computing resource(s) 114 may determine a likelihood that a medical professional will use the supporting evidence associated with the recommendation. For example, the remote computing resource(s) 114 may determine a statistical relevance of the supporting evidence and may determine the likelihood that the medical professional will utilize the supporting evidence based on the statistical relevance of the supporting evidence. In some cases, the remote computing resource(s) 114 may transmit the recommendation(s) and/or the supporting evidence associated with the recommendation(s) to the device 106 and may cause the device 106 to present the supporting evidence to the medical professional based on determining the likelihood that the supporting evidence will be utilized. For example, the supporting evidence may be presented in an order listed from most likely to be utilized to least likely to be utilized (e.g., in the case of a diabetes diagnosis, present a blood sugar test ahead of a skin biopsy test). That is, the remote computing resource(s) may rank the supporting evidence based on a likelihood that the supporting evidence will be utilized and present the supporting evidence in a list based on the ranking. In some cases, the remote computing resource(s) 114 may cause the remote device to emphasize (e.g., highlighted, bolded, italicized, underlined, etc.) supporting evidence that is more likely to be utilized. In this way, the medical professional can quickly determine if the recommendation(s) provided are accurate and the medical professional can efficiently and swiftly attend to the patient.

The remote computing resource(s) 114 are configured to receive, from the device 106, prompts, messages, feedback, or response(s) (e.g., words, phrase, sentences, selections etc.) to the indicate which supporting evidence was used to determine if the recommendation is accurate. In some instances, the feedback may be received by a feedback engine 212 and/or the processor(s) 118 may forward the feedback to the historical records 208. Upon receiving the feedback, the feedback engine 212 may be configured to analyze the feedback to determine words, phrases, and expressions contained therein. For instance, the feedback may include an indication of which of the supporting evidence was used to determine that the recommendation was accurate. Therein, the prediction analytics component 200 may utilize the feedback to help predict outcomes, correlations, or other relationships that indicate a likelihood that the medical professional will utilize certain types of information.

In one example of generating and transmitting a recommendation, the prediction analytics component 200 may utilize the user profile(s) 122, the medical professional profiles(s) 124, and/or the database(s) 126 to determine that “150” is a normal and/or healthy blood sugar level after eating. In some instances, this determination may result from comparing the value with the user profile(s) 122 and/or the database(s) 126. For instance, the prediction analytics component 200 may compare “150 mg/dL” to determine that other patients having this blood sugar level were not diagnosed with diabetes, thereby utilizing correlations between other patients and their symptoms.

Noted above, certain symptoms may be weighed by the prediction analytics component 200 in determining the recommendation(s) and supporting evidence associated with the recommendation. For instance, a blood sugar level of 240 mg/dL may be weighed more heavily in determining a probability of the patient being diabetic, as compared to whether the patient is experienced blurred vision.

The prediction analytics component 200 may also reference other diagnoses and/or systems stored in other user profile(s) 122. In this sense, the prediction analytics component 200 may compare symptoms of a respective patient with symptoms experienced by other patients in determining suspected diagnoses and mapping the user profile(s) 122 together and analyzing trends. For instance, other patients may have experienced similar symptoms as the patient and the prediction analytics component 200 may use these indications to determine suspected diagnoses of the patient. In some instances, the amount of influence this factor has may decay over time. For instance, if two patients are experiencing similar symptoms and one was diagnosed with diabetes within a year, then the prediction analytics component 200 may weight this interaction more greatly than if the diagnosis was several years prior.

The user profile(s) 122, the medical professional profile(s) 124, and/or the database(s) 126 may be updated based on the recommendation(s) and supporting evidence associated with the recommendation, such as symptoms indicated by the recommendations. Additionally, in some examples, the remote computing resource(s) 114 may obtain, retrieve, and/or receive the medical history 202, the personal information 204, the historical records 208, and/or the third-party medical data 206 continuously from the third-party sources. In some examples, the remote computing resource(s) 114 may obtain, retrieve, and/or receive the medical history 202, the personal information 204, the historical records 208, and/or the third-party medical data 206 at given time intervals. The given time intervals may include, but are not limited to, every minute, half-hour, hour, day, week, month, or the like.

Additionally, to protect the privacy of information contained in the user profile(s) 122, the remote computing resource(s) 114 may receive consent from patients to share, correlate, or otherwise use the information in determine one or more suspected diagnoses. That is, as noted above, the remote computing resource(s) 114 may correlate symptoms of one patient with symptoms or another patient in determining suspected diagnoses, recommendation(s), and supporting evidence associated with the recommendation. Before such correlation of comparisons, the remote computing resource(s) 114 may first receive consent.

### Illustrative Device

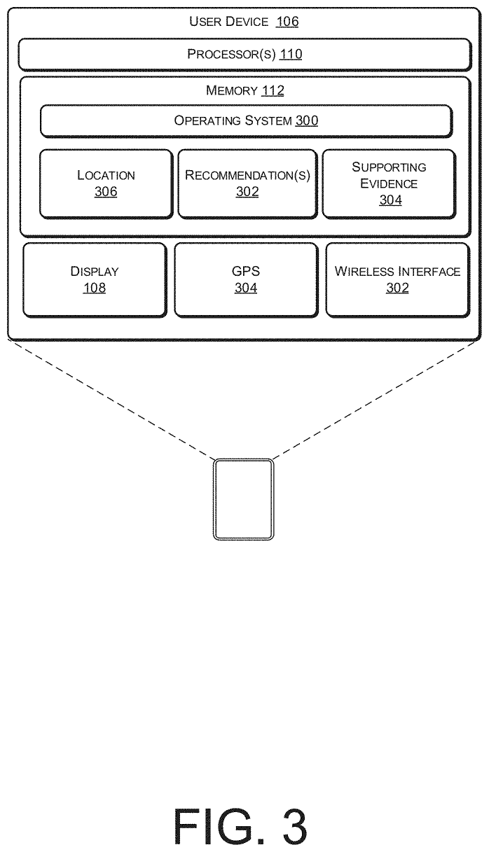

FIG. 3 shows selected functional components of the device 106. Generally, the device 106 may be implemented as a standalone device that is relatively simple in terms of functional capabilities with input/output components, memory (e.g., the memory 112), and processing capabilities. For instance, the device 106 may include the display 108 or a touchscreen to facilitate visual presentation (e.g., text, charts, graphs, images, etc.), graphical outputs, and receive user input through either touch inputs on the display 108 (e.g., virtual keyboard).

The memory 112 stores an operating system 300. The operating system 300 may configure the processor(s) 110 to display recommendation(s) 302 and supporting evidence 304 associated with the recommendation(s) 302 on the display 108. Display of the recommendation(s) 302 and supporting evidence 304 associated with the recommendation(s) 302 may involve displaying selectable text where a user (e.g., provider 102) is able to provide input, as shown and discussed below in FIG. 6. In some instances, multiple recommendation(s) 302 and supporting evidence 304 associated with the recommendation(s) 302 may be displayed in unison, or at the same time on the display 108, or only one recommendation(s) 302 and supporting evidence 304 associated with the recommendation(s) 302 may be presented at a time on the display 108. Further, the device 106 may be configured to transmit one or more user input at the same time, or user input may be submitted individually.

In the illustrated example, the device 106 includes a wireless interface 306 to facilitate a wireless connection to a network (e.g., the network 116) and the remote computing resource(s) 114. The wireless interface 306 may implement one or more of various wireless technologies, such as WiFi, Bluetooth, RF, and the like.

FIG. 3 also illustrates that the device 106 may include global positioning systems (GPS) 308 or other locating devices may be used. The GPS 308 may generate a location 310 that corresponds to a location of the device 106. In some instances, the processor(s) 110 may utilize the location 310 in downloading or receiving the recommendation(s) 302 and supporting evidence 304 associated with the recommendation(s) 302 from the remote computing resource(s) 114. For instance, the location 310 may indicate that the device 106 is within a residence of a patient or a threshold proximity thereof. In response, the processor(s) 110 may receive (e.g., download) the recommendation(s) 302 and supporting evidence 304 associated with the recommendation(s) 302 from the remote computing resource(s) 114. In another instance, the location 310 may indicate the device 106 is traveling towards the residence of the patient, and in response, the device 106 may receive the recommendation(s) 302 and supporting evidence 304 associated with the recommendation(s) 302. As noted above, however, to receive the recommendation(s) 302 and supporting evidence 304 associated with the recommendation(s) 302, the processor(s) 110 may transmit a pull request, or the remote computing resource(s) 114 may push the recommendation(s) 302 and supporting evidence 304 associated with the recommendation(s) 302 in response to determining the device 106 is within the residence or is in route to the patient's residence.

In some instances, the device 106 may include one or more microphones that receive audio input, such as voice input from the provider 102 and/or the patient 104, and one or more speakers to output audio. For instance, the provider 102 or the patient 104 may interact with the device 106 by speaking to it, and the one or more microphone captures the user speech. In response, the device 106 performs speech recognition (e.g., speech recognition engine and/or speech-to-text) and types text data into a field corresponding to the speech. Additionally, or alternatively, the audio data may be provided to the remote computing resource(s) 114 as user input, where the remote computing resource(s) 114 analyzes the user input. To relay the recommendation(s) 302 and supporting evidence 304 associated with the recommendation(s) 302 to the patient 104, the device 106 may emit audible statements through the speaker. In this manner, and in some instances, the provider 102 and/or the patient 104 may interact with the device 106 through speech, without using and/or in addition to the virtual keyboard presented on the display 108, for instance.

In some instances, the memory 112 may include the user profile(s) 122, the medical professional profile(s) 124, the databases 126, the prediction analytics component 200, and/or the feedback engine 212. Additionally, at least some of the processes of the remote computing resource(s) 114 may be executed by the device 106.

### Illustrative Processes

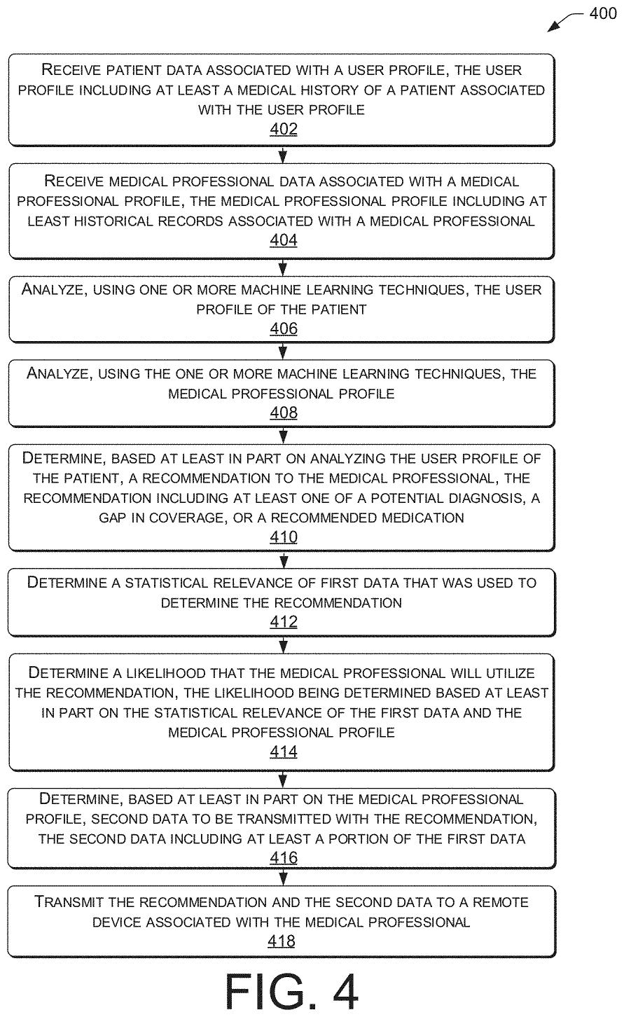

FIG. 4 illustrates various processes related to for surfacing supporting evidence associated with recommendations. The processes described herein are illustrated as collections of blocks in logical flow diagrams, which represent a sequence of operations, some or all of which may be implemented in hardware, software, or a combination thereof. In the context of software, the blocks may represent computer-executable instructions stored on one or more computer-readable media that, when executed by one or more processors, program the processors to perform the recited operations. Generally, computer-executable instructions include routines, programs, objects, components, data structures and the like that perform particular functions or implement particular data types. The order in which the blocks are described should not be construed as a limitation, unless specifically noted. Any number of the described blocks may be combined in any order and/or in parallel to implement the process, or alternative processes, and not all of the blocks need be executed. For discussion purposes, the processes are described with reference to the environments, architectures and systems described in the examples herein, such as, for example those described with respect to FIGS. 1-3 and 6, although the processes may be implemented in a wide variety of other environments, architectures and systems.

FIG. 4 illustrates a process 400 for determining recommendations and providing supported evidence associated with the recommendations. At block 402, the process 400 may receive patient data associated with a user profile, the user profile including at least a medical history of a patient associated with the user profile. For instance, user profile(s) 122 may correspond to a respective user (e.g., patients). Each user profile 122 may include a user's medical history 202 and personal information 204. In some instances, the medical history 202 may include a medical history of the user, such as diagnoses (e.g., disease, illness, etc.), treatments (e.g., medications, surgeries, therapy, etc.), family medical history (e.g., diabetes, Alzheimer's, etc.), measurements (e.g., weight, height, etc.), symptoms (e.g., sore throat, back pain, loss of sleep, etc.), and so forth. The personal information 204 may include names (e.g., social security number (SSN)), identifiers, residence, work history, acquaintances, heritage, age, and so forth. The medical history 202 and/or the personal information 204 may be received using record locators and/or searching databases.

At block 404, the process 400 may receive medical professional data associated with a medical professional profile, the medical professional profile including at least historical records associated with a medical professional. For instance, the prediction analytics component 200 may access the medical professional profile 124 and a historical record(s) 208 and determine which types of information (i.e., supporting evidence) that a particular medical professional commonly uses to determine if a recommendation is accurate. This may be done by receiving feedback from the medical professional indicating which information included in the supporting evidence was used to determine if the recommendation(s) is accurate.

At block 406, the process 400 may analyze, using one or more machine learning techniques, the user profile of the patient. For instance, the remote computing resource(s) 114 may analyze the user profile(s) 122 and/or the database(s) 126. The analysis at block 406 may be performed by the prediction analytics component 200 discussed hereinabove. In some instances, the block 402 may be performed in response to certain actions, such as a patient requesting an examination and/or a patient enrolling in a new health care plan.

At block 408, the process 400 may analyze, using the one or more machine learning techniques, the medical professional profile. For instance, the prediction analytics component 200 may access the medical professional profile 124 and a historical record(s) 208 and determine which types of information (i.e., supporting evidence) that a particular medical professional commonly uses to determine if a recommendation is accurate. This may be done by receiving feedback from the medical professional indicating which information included in the supporting evidence was used to determine if the recommendation(s) is accurate.

At block 410, the process 400 may determine, based at least in part on analyzing the user profile of the patient, a recommendation to the medical professional, the recommendation including at least one of a potential diagnosis, a gap in medical coverage, or a recommended medication. For instance, the remote computing resource(s) 114 may analyze the user profile(s) 122 and/or the database(s) 126 to generate recommendation(s) and supporting evidence associated with the recommendation(s) for a patient. For instance, the prediction analytics component 200 may analyze the user profile(s) 122, the medical professional profile(s) 124, and/or the database(s) 126 to determine recommendation(s) and supporting evidence associated with the recommendation(s). The prediction analytics component 200 may also be configured to determine a statistical relevance of individual data included in the supporting relevance and a likelihood that a particular medical professional, such as provider 102, will utilize the supporting evidence when determining the accuracy of the recommendation. Stated alternatively, the prediction analytics component 200 functions to determine recommendations, such as suspected diagnoses of the patient (e.g., diabetes, heart disease, etc.), potential gaps in coverage (e.g., mammograms) associated with the patient, and/or a recommended prescription for the patient that should be asked of the patient in determining one or more suspected health concerns (or diagnoses) of the patient or whether the patient is suspected of having particular diagnoses.

At block 412, the process 400 may determine a statistical relevance of first data that was used to determine the recommendation. For instance, the prediction analytics component 200 may determine the statistical relevance of the information used to determine the recommendation(s) by comparing the recommendation(s) and information used to determine recommendation(s) to previous recommendation(s) and previous information used to determine recommendation(s). In some instances, the prediction analytics component 200 may access the medical professional profile 124 and a historical record(s) 208 and determine which types of information (i.e., supporting evidence) that a particular medical professional commonly uses to determine if a recommendation is accurate. This may be done by receiving feedback from the medical professional indicating which information included in the supporting evidence was used to determine if the recommendation(s) is accurate. In some instances, the prediction analytics component 200 may utilize a machine learning model to determine which information is most statistically relevant by determining a confidence score of the recommendation. For example, a recommendation based off a first test, a second test, and a third test may result in a 95% confidence score of the recommendation, via a machine learning model. The prediction analytics component 200 may determine that removal of the third test from the machine learning model results in a 94% confidence score of the recommendation (i.e., the recommendation being based off of the first test and the second test) and removal of the second confidence score results in a 50% confidence score of the recommendation (i.e., the recommendation being based off of the first test and the third test). The remote computing resource(s) may then determine that the second test is more statistically relevant than the third test due to the effect it has on the confidence score of the recommendation. In some instances, removal of a single particular test may have a minimal effect on the confidence score of the recommendation, but removal of multiple tests may have a substantial effect on the confidence score of the recommendation. In this case, the prediction analytics component 200 may determine that the multiple tests are substantially equally statistically relevant.

At block 414, the process 400 may determine a likelihood that the medical professional will utilize the recommendation, the likelihood being determined based at least in part on the statistical relevance of the first data and the medical professional profile. For instance, the remote computing resource(s) 114 may determine a likelihood that a medical professional will use the supporting evidence associated with the recommendation. For example, the remote computing resource(s) 114 may determine a statistical relevance of the supporting evidence and may determine the likelihood that the medical professional will utilize the supporting evidence based on the statistical relevance of the supporting evidence. In some cases, the remote computing resource(s) 114 may determine that the medical professional will utilize the recommendation based on previous interactions that the medical professional has had in interacting with the remote computing resource(s) 114. For example, the remote computing resource(s) 114 may store feedback received from the medical professional from previous interactions in the historical records 208. The remote computing resource(s) 114 may access the historical records 208 to determine which types of information that the medical professional has previously utilized to determine an accuracy of a recommendation and the remote computing resource(s) 114 may determine that similar types of information are present in the first data. In one example, the remote computing resource(s) 114 may determine that in a previous instance the medical professional utilized a certain test from “Company A” instead of the same type of test from “Company B.” In this case, the remote computing resource(s) 114 may determine that the medical professional is more likely to utilize information from “Company A” as opposed to information from “Company B.”

At block 416, the process 400 may determine, based at least in part on the medical professional profile, second data to be transmitted with the recommendation, the second data including at least a portion of the first data. For instance, the remote computing resource(s) 114 may transmit the recommendation(s) and/or the supporting evidence associated with the recommendation(s) to the device 106 and may cause the device 106 to present the supporting evidence to the medical professional based on determining the likelihood that the supporting evidence will be utilized. For example, the supporting evidence may be presented in an order listed from most likely to be utilized to least likely to be utilized (e.g., in the case of a diabetes diagnosis, present a blood sugar test ahead of a skin biopsy test). In some cases, the remote computing resource(s) 114 may cause the remote device to emphasize (e.g., highlighted, bolded, italicized, underlined, etc.) supporting evidence that is more likely to be utilized. In this way, the medical professional can quickly determine if the recommendation(s) provided are accurate and the medical professional can efficiently and swiftly attend to the patient.

At block 418, the process 400 may transmit the recommendation and the second data to a remote device associated with the medical professional. In some instances, the recommendation(s) and/or the supporting evidence associated with the recommendation(s) may be transmitted to the device 106 in response to a pull request from the device 106. Additionally, or alternatively, the recommendation(s) and/or the supporting evidence associated with the recommendation(s) may be pushed to the device 106 after generating the recommendation(s) and/or the supporting evidence associated with the recommendation(s). The remote computing resource(s) 114 may transmit the recommendation(s) and/or the supporting evidence associated with the recommendation(s) with a command that causes the device 106 to display the recommendation(s) and/or the supporting evidence associated with the recommendation(s). To communicate with the device 106, the third-party sources providing the third-party data 206, or other entities, the remote computing resource(s) 114 include an interface 210.

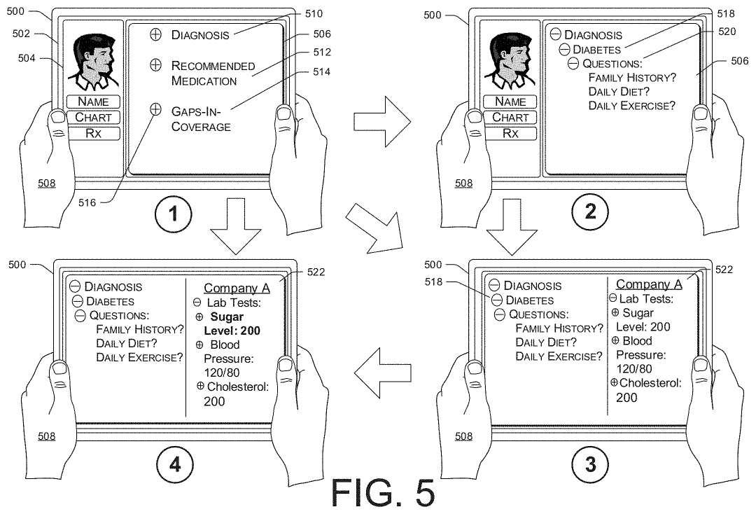

FIG. 5 illustrates an iterative process of displaying recommendations(s) and supporting evidence associated with the recommendation(s) on a device 500 (which may be similar to and/or represent the device 106). The progression of the process shown in FIG. 5 is illustrated by the arrows.

The device 500 is shown including a display 502 having a first area 504 and a second area 506. In the first area 504, background information of a patient is displayed. For instance, the first area 504 may include an image of the patient, a name of the patient, medical charts of the patient, or prescriptions of the patient. However, while FIG. 5 illustrates certain background information, other information may be displayed as well, or the background information may be presented differently than shown. The background information may be accessed through a user 508 interacting with the display 502. For instance, the user 508 may select “Chart” within the first area and medical charts of the patient may be displayed on the display 502.

Shown at “1,” the second area 506 displays a number of recommendation(s) that may be selectable by the user 508. In this example, the recommendations include a diagnosis 510, a recommended medication 512, and a potential gaps-in-coverage 514. The user 508 may expand any one of the recommendation(s) to view additional information associated with the recommendation(s). For example, each recommendation may include a selectable option 516 which causes the device 500 to present additional information.

As shown at “2,” the device 500 displays a proposed diagnosis 518 in response to the user 508 selecting the diagnosis 510 recommendation. Additionally, in some cases, the device 500 may also display a number of questions 520 that are associated with the proposed diagnosis 518. The questions 520 are intended for the user 508 to ask the patient in order to aid in the medical care provided to the patient. In the example shown on the device 500, the questions include asking the patient questions regarding “Family History?”, “Daily Diet?”, and “Daily Exercise?”. It is understood that other questions associated with the proposed diagnosis 518 may be included.

As shown at “3”, the device 500 displays a number of supporting evidence 522 that are associated with the proposed diagnosis 518. The information displayed at “3” may be presented after the information displayed at “2” or may be display after the information displayed at “1”. In some cases, the device 500 may display the supporting evidence 522 in response to the user 508 selecting one of the recommendations display at “1.” As discussed above, the supporting evidence 522 may include any information that is associated with the recommendation(s), and this case, the proposed diagnosis 518 (i.e., “diabetes”). In this case, the supporting evidence 522 includes a number of tests that were performed (i.e., “sugar level: 200,” “blood pressure 12/80,” and “cholesterol: 200”) as well as the name of the company that performed the test (i.e., “Company A”). It is understood that the supporting evidence 522 may include more information or less information than is displayed on device 500. For example, the supporting evidence 522 may include information from a number of different companies, medical history, personal information, and/or from a number of different tests. Furthermore, the supporting evidence 522 may be listed in a particular order based on a statistical relevance and/or a likelihood that the user 508 will find the information relevant to the proposed diagnosis 518. For example, in the case of the proposed diagnosis 518 being “diabetes,” the supporting evidence 522 may list the “sugar level: 200” before the “blood pressure 120/80” and the “cholesterol: 200” because the “sugar level: 200” is more relevant to the “diabetes” proposed diagnosis 518 than “blood pressure 120/80” and the “cholesterol: 200.” As shown at “4”, the supporting evidence 522 may emphasize (e.g., highlighted, bolded, italicized, underlined, etc.) a particular set of information included in the supporting evidence based on a statistical relevance and/or a likelihood that the user 508 will find the information relevant to the proposed diagnosis 518. For example, in the case of the proposed diagnosis 518 being “diabetes,” the supporting evidence 522 may bold the “sugar level: 200” and not bold “blood pressure 120/80” and the “cholesterol: 200” because the “sugar level: 200” is more relevant to the “diabetes” proposed diagnosis 518 than “blood pressure 120/80” and the “cholesterol: 200.” The information displayed at “4” may be presented after the information displayed at “3” or may be display after the information displayed at “1”. In some cases, the device 500 may display the supporting evidence 522 at “4” in response to the user 508 selecting one of the recommendations display at “1.” At either of “3” or “4” the user 508 may select any of the supporting evidence 522 to indicate that the particular piece of supporting evidence 522 is being utilized to determine that the propose4d diagnosis 518 is accurate. This type of feedback may be sent to servers, such as the remote computing resource(s) 114, that are causing presentation of the data on the device 500.

### CONCLUSION

While the foregoing invention is described with respect to the specific examples, it is to be understood that the scope of the invention is not limited to these specific examples. Since other modifications and changes varied to fit particular operating requirements and environments will be apparent to those skilled in the art, the invention is not considered limited to the example chosen for purposes of disclosure and covers all changes and modifications which do not constitute departures from the true spirit and scope of this invention.

Although the application describes embodiments having specific structural features and/or methodological acts, it is to be understood that the claims are not necessarily limited to the specific features or acts described. Rather, the specific features and acts are merely illustrative some embodiments that fall within the scope of the claims of the application.

What is claimed is:

1. A system comprising:
one or more processors; and
non-transitory computer-readable media storing first computer-executable instructions that, when executed by the one or more processors, cause the one or more processors to perform operations comprising:
receiving patient data associated with a user profile, the user profile including at least a medical history of a patient associated with the user profile;
receiving medical professional data associated with a medical professional profile, the medical professional profile including at least historical records associated with a medical professional;
analyzing, using one or more machine learning techniques, the user profile;
analyzing, using the one or more machine learning techniques, the medical professional profile;
determining, based at least in part on analyzing the user profile, a recommendation to the medical professional, the recommendation including at least one of a potential diagnosis, a gap in medical coverage, or a medication-related recommendation;
determining a statistical relevance of data utilized for determining the recommendation;
determining a likelihood that the medical professional will utilize the data in association with the recommendation, the likelihood being determined based at least in part on the statistical relevance of the data and the medical professional profile;
transmitting the recommendation and the data to a remote device associated with the medical professional.

2. The system of claim 1, the operations further comprising ranking at least one of the potential diagnosis, the gap in medical coverage, or the recommended medication based at least in part on the likelihood that the medical professional will utilize the recommendation, wherein the recommendation is transmitted based at least in part on the ranking.

3. The system of claim 1, wherein determining the likelihood that the medical professional will utilize the data includes determining that the medical professional has utilized previous data that is associated with the data.

4. The system of claim 1, the operations further comprising receiving an indication that the patient is scheduled to meet with the medical professional at a given time and causing the remote device to display the recommendation and the data at the given time.

5. The system of claim 4, wherein the user interface includes a first section for presenting the recommendation and a selectable portion that, in response to being selected, causes a second section to present content corresponding to the data, the first section being adjacent to the second section.

6. The system of claim 1, wherein the data that was used to determine the recommendation includes at least one of a test result, medical history, personal information, or identifying information associated with a test results.

7. The system of claim 1, wherein determining the statistical relevance of the data is based at least in part on a degree of change that the data has on a confidence score associated with the recommendation.

8. The system of claim 1, wherein the data comprises first data and the operations further comprising:
determining that a first portion of the first data is more relevant than a second portion of the first data;
generating second data including the second portion of the first data, the second data including content that, when displayed, includes at least an emphasized portion; and
causing the remote device to display the second data.

9. A method comprising:
receiving patient data associated with a user profile, the user profile including at least a medical history of a patient associated with the user profile;
receiving medical professional data associated with a medical professional profile, the medical professional profile including at least historical records associated with a medical professional;
analyzing, using one or more machine learning techniques, the user profile;
analyzing, using the one or more machine learning techniques, the medical professional profile;
determining, based at least in part on analyzing the user profile, a recommendation to the medical professional;
determining, based at least in part on the recommendation, data to be transmitted with the recommendation;
determining a likelihood that the medical professional will utilize the data in association with the recommendation;
transmitting the recommendation and the data to a remote device associated with the medical professional.

10. The method of claim 9, wherein the recommendation includes, at least one of a potential diagnosis, a gap in medical coverage, or a medication related recommendation.

11. The method of claim 9, wherein determining the likelihood that the medical professional will utilize the recommendation is based at least in part on a statistical relevance of the data utilized for determining the recommendation.

12. The method of claim 11, further comprising ranking the data based at least in part on the likelihood that the medical professional will utilize the recommendation, wherein the data is transmitted based at least in part on the ranking.

13. The method of claim 11, wherein the statistical relevance of the data is based at least in part on a degree of change that the data has on a confidence score associated with the recommendation.

14. The method of claim 9, wherein determining the data includes determining that the medical professional has utilized previous data that is associated with the data.

15. The method of claim 9, wherein the data comprises first data and the operations further comprising:
determining that a first portion of the first data is more relevant than a second portion of the first data;
generating second data including the second portion of the first data, the second data including content that, when displayed, includes at least an emphasized portion; and
causing the remote device to display the second data.

16. A system comprising:
at least one processor; and
one or more non-transitory computer-readable media storing first computer-executable instructions that, when executed by the at least one processor, cause the at least one processor to perform acts comprising:
receiving patient data associated with a user profile, the user profile including at least a medical history of a patient associated with the user profile;
receiving medical professional data associated with a medical professional profile, the medical professional profile including at least historical records associated with a medical professional;
analyzing, using one or more machine learning techniques, the user profile;
analyzing, using the one or more machine learning techniques, the medical professional profile;
determining, based at least in part on analyzing the user profile, a recommendation to the medical professional;
determining, based at least in part on the recommendation, data to be transmitted with the recommendation;
determining a likelihood that the medical professional will utilize the data in association with the recommendation;
transmitting the recommendation and the data to a remote device associated with the medical professional.

17. The system of claim 16, wherein the recommendation includes, at least one of a potential diagnosis, a gap in medical coverage, or a medication related recommendation.

18. The system of claim 16, wherein determining the likelihood that the medical professional will utilize the recommendation is based at least in part on a statistical relevance of the data utilized for determining the recommendation.

19. The system of claim 16, the operations further comprising receiving an indication that the patient is scheduled to meet with the medical professional at a given time and causing the remote device to display the recommendation and the data at the given time.

20. The system of claim 19, wherein the user interface includes a first section for presenting the recommendation and a selectable portion that, in response to being selected, causes a second section to present content corresponding to the data, the first section being adjacent to the second section.
